(19) United States
(12) Patent Application Publication
(10) Pub. No.: US 2013/0023024 A1
(43) Pub. Date: Jan. 24, 2013

(54) CONJUGATE OF MAGNETIC PARTICLE AND SURFACE MODIFIER LINKED THROUGH CLEAVABLE PEPTIDE BOND

(71) Applicants: Jackie Y. Ying, Singapore (SG); Karthikeyan Narayanan, Singapore (SG); Alex Wei Haw Lin, Singapore (SG); Andrew Chwee Aun Wan, Singapore (SG); Yuangang Zheng, Singapore (SG); Nandanan Erathodiyil, Singapore (SG)

(72) Inventors: Jackie Y. Ying, Singapore (SG); Karthikeyan Narayanan, Singapore (SG); Alex Wei Haw Lin, Singapore (SG); Andrew Chwee Aun Wan, Singapore (SG); Yuangang Zheng, Singapore (SG); Nandanan Erathodiyil, Singapore (SG)

(21) Appl. No.: 13/637,524

(22) PCT Filed: Mar. 31, 2011
(86) PCT No.: PCT/SG11/00134

§ 371 Date: Sep. 26, 2012

(30) Foreign Application Priority Data
Mar. 31, 2010 (SG) 201002272-1

(51) Int. Cl. C12N 13/00; C12N 5/071; C07K 7/06; B82Y 99/00

(52) U.S. Cl. 435/173.9; 530/322; 435/325; 977/774; 977/773

(57) ABSTRACT

A conjugate is provided for cell processing, which comprises a magnetic particle and a surface modifier having specific affinity to a target cell. The particle and modifier are linked through a cleavable peptide bond. In a method of cell processing, the conjugate is attached to a target cell; the target cell attached to the conjugate is subject to magnetic processing; the peptide bond is cleaved to separate the processed target cell from the magnetic particle; the target cell separated from the magnetic particle is attached to a substrate. The magnetic particle may include an iron oxide, and the surface modifier may include a glucosamine. The particle and modifier may be linked by a linker comprising a protease recognition site and a peptide bond. The linker links the surface modifier to the particle, and cleavage of the peptide bond is catalyzed by a specific protease that recognizes the protease recognition site.

### CROSS-REFERENCE TO RELATED APPLICATION

This application claims the benefit of and priority from Singapore Patent Application No. 2010002272-1, filed Mar. 31, 2010 and entitled “Glucosamine-conjugated Iron Oxide Nanoparticles for the Separation of Insulin Secreting Beta Cells,” the entire contents of which are incorporated herein by reference.

### FIELD OF THE INVENTION

The present invention relates to conjugates for cell manipulation and processing, use of the conjugates, and methods of cell manipulation and processing.

### BACKGROUND OF THE INVENTION

The movement of cells may be controlled by binding magnetic particles to target cells and applying a magnetic field to move the magnetic particles and thus the target cells bonded to the magnetic particles. Such techniques may be used in cell processing, such as cell manipulation, cell separation, cell sorting, or other applications where control of cell movement is needed. Such techniques are thus useful in a wide variety of biomedical applications, tissue engineering, and other processes involving the use of cells. For example, cell separation may be used to remove unwanted cells, to collect desired cells, to purify a cell population, or to control the cell environment. Magnetic particles bonded to cells may also be used to mark or label cells for cell detection or magnetic imaging.

Cellular adhesion is the binding of a cell to a surface, extracellular matrix or another cell, typically mediated by cell adhesion molecules such as cell surface proteins that are selectins, integrins, or cadherins. Cellular adhesion is an aspect of cellular growth and multiplication for many cell types (Gumbiner, B. M., “Cell adhesion: The molecular basis of tissue architecture and morphogenesis,” Cell, (1996), vol. 84, pp. 345-357).

### SUMMARY OF THE INVENTION

In one aspect, the invention provides a conjugate that may be used to facilitate the magnetic processing of cells, the conjugate having a linker that may be cleaved to facilitate subsequent cellular processes, such as cellular adhesion. In selected embodiments, a conjugate disclosed herein comprises a magnetic particle and a surface modifier having a specific affinity to target cells. The particle and the modifier are linked through a cleavable peptide bond specific to a protease.

The conjugates can attach to the target cells and can be used for cell processing, such as cell sorting or cell separation with magnetic force, and magnetic imaging or detection. The magnetic particles can be conveniently separated from the target cells after initial processing and before attaching the cells to a substrate, by exposing the processed target cells to the specific protease to cleave the peptide bonds, thus severing the links between the magnetic particles and the cells. Subsequently, the target cells separated from the magnetic particles can be conveniently attached to the substrate, without interference from the magnetic particles.

Thus, in accordance with an aspect of the present invention, there is provided a conjugate comprising a magnetic particle comprising an iron oxide; a surface modifier comprising a glucosamine; and a linker comprising a protease recognition site and a peptide bond. The linker links the surface modifier to the particle, and cleavage of the peptide bond is catalyzed by a specific protease that recognizes the protease recognition site. The protease may be thrombin. The magnetic particle may comprise a quantum dot. The particle may be a nanoparticle. The particle may be superparamagnetic. The particle may comprise magnetite. The linker may comprise a protease recognition sequence. The protease recognition sequence may comprise Leu-Val-Pro-Arg-Gly-Ser.

In accordance with a further aspect of the present invention, there is provided a method of forming a conjugate as described in the preceding paragraph, comprising linking the surface modifier to the magnetic particle with the linker.

In accordance with another aspect of the present invention, there is provided a method of cell processing. In this method, a conjugate is attached to a target cell, where the conjugate comprises a magnetic particle and a surface modifier having a specific affinity to the target cell. The particle and modifier are linked through a cleavable peptide bond. The target cell attached to the conjugate is then subject to magnetic processing. The peptide bond is cleaved to separate the target cell from the magnetic particle. A substrate is provided and the target cell separated from the magnetic particle is allowed to attach to the substrate. The conjugate may comprise a linker linking the surface modifier to the magnetic particle, wherein the linker comprises a protease recognition site and the peptide bond, and cleavage of the peptide bond is catalyzed by a specific protease that recognizes the protease recognition site. Cleaving the peptide bond may comprise exposing the linker to the protease. The protease may be thrombin. The surface modifier may comprise a glucosamine, glutamine, or galactose. The magnetic particle may comprise .a quantum dot or a nanoparticle. The magnetic particle may be superparamagnetic. The magnetic processing may comprise magnetically sorting or separating cells. The conjugate may be any conjugate disclosed herein.

In accordance with a further aspect of the present invention, there is provided a method of forming a conjugate for attachment to a cell. The method comprises linking a surface modifier to a magnetic particle through a linker to form the conjugate. The surface modifier is selected to have a specific affinity to the cell. The linker is selected such that it comprises a protease recognition site and a peptide bond, and cleavage of the peptide bond is catalyzed by a specific protease that recognizes the protease recognition site. The protease may be thrombin. The surface modifier may comprise a glucosamine, glutamine, or galactose. The magnetic particle may comprise a quantum dot or a nanoparticle. The conjugate may be any conjugate disclosed herein.

In accordance with another aspect of the present invention, a conjugate disclosed herein is used in the processing of cells, such as magnetically sorting or separating the cells.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a schematic diagram of a conjugate, exemplary of an embodiment of the present application;

FIG. 2 is a schematic diagram of a chemical reaction for forming maleimidoglucosamine;

FIG. 3 is a schematic diagram of a chemical reaction for forming a glucosamine-peptide complex;

FIG. 4 is a flow chart for a process of cell separation, exemplary of an embodiment of the present application;

FIG. 5 is a flow chart for cell processing, exemplary of an embodiment of the present application;

FIG. 6 is a schematic diagram for the synthesis route of forming comparison conjugates;

FIG. 7 is a transmission electron microscopy (TEM) image of the comparison conjugates formed according to the synthesis route of FIG. 6;

FIG. 8 is a TEM image of sample iron oxide nanoparticles used for forming the conjugates of FIG. 7;

FIG. 9 is a dynamic light scattering (DLS) spectrum for sample nanoparticles of FIG. 8;

FIGS. 10, 11 and 12 are confocal microscopic images of sample cells with different attachments;

FIGS. 13 and 14 are data graphs showing cell uptake in different sample mixtures;

FIG. 15 is bar graph showing real-time polymerase chain reaction (PCR) test results for different sample mixtures;

FIG. 16 is a bar graph showing real-time PCR results of sample cells attached to the conjugates of FIG. 7;

FIG. 17 is a line graph showing different binding affinities of different cells to the conjugates of FIG. 7;

FIG. 18 is a data graph showing the results of flow cytometry analysis of sample mixture of cells prior to cell separation;

FIG. 19 is a data graph showing the results of flow cytometry analysis of the flown-through fraction of the sample mixture of FIG. 18 after cell separation;

FIG. 20 is a data graph showing the results of flow cytometry analysis of the conjugate-bonded fraction of the sample mixture of FIG. 18 after cell separation;

FIG. 21 is a bar graph showing real-time PCR results of sample cells;

FIG. 22 is a bar graph showing the percentage of cells in samples incubated with conjugates having peptide linker and conjugates having no peptide linker respectively; and

FIGS. 23 and 24 are images of culture substrates after cell culture with the respective sample cells of FIG. 22.

### DETAILED DESCRIPTION

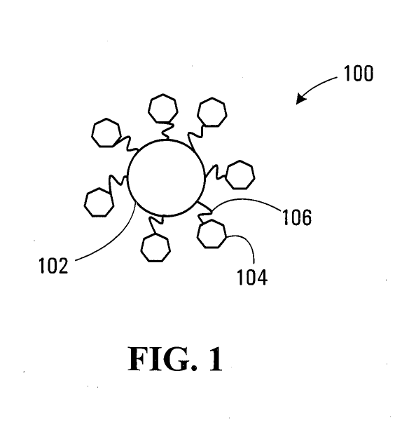

An exemplary embodiment of the present invention is a conjugate 100 of a magnetic particle 102 and a surface modifier 104, as illustrated in FIG. 1. Particle 102 and modifier 104 are linked by a severable linker 106.

Magnetic particle 102 may be a nanoparticle. Nanoparticles typically refer to particles having a particle size of about 1 to about 100 nm. In some embodiments, particle 102 may have a particle size of about 6 to about 8 nm. In alternative embodiments, the particle size may be from about 2 to about 20 nm. In further embodiments, the particle size may be about 50 nm. The particle size may also be larger, such as from about 100 nm to a few micrometers. In one embodiment, the particle size may be about 150 nm, or larger than 2 μm. Other particle sizes may also be selected depending on the particular application. Particle 102 may have any shape, such as a generally spherical, generally cubic, or irregular shape. In some applications, the shapes and sizes of the particles used may be substantially uniform, and may be controlled for a particular purpose. In other applications, the sizes or shapes of the particles may vary. The term “particle size” as used herein refers to the average diameter of the particle when the particle has a generally spherical shape. As particles may have non-spherical shapes and different sizes, the particle size refers to the average size of the particles when used in reference to multiple particles. When a particle has an irregular non-spherical shape, its particle size refers to its effective diameter, which is the diameter of a spherical particle that has the same volume as the non-spherical particle. In cases where the particle has a generally geometrical shape, such as a cubic shape, the particle size may refer to a characteristic dimension for that geometrical shape. For example, a cubic shape may be characterized by the length of its side.

Particle sizes and size distribution of particles can be measured using optical or electronic imaging techniques, such as transmission electron microscopy (TEM) or suitable light scattering (e.g. dynamic light scattering) techniques. Such techniques can be readily understood and applied by persons skilled in the art for a given application. The average particle size may be determined using standard techniques, for example, by measuring the size of a representative number of particles.

Particle 102 is formed of a magnetic material such that its movement can be controlled by applying a magnetic force, the benefits of which will become apparent below. The magnetic material may be ferromagnetic, or superparamagnetic. In some embodiments, particle 102 may be formed of an iron oxide, such as magnetite (Fe₃O₄). As can be appreciated, magnetite is more magnetic and magnetite particles may be conveniently manipulated with a weaker magnetic force, as compared to particles formed of other forms of iron oxides. However, in some embodiments, other forms of magnetic iron oxides may also be used. Possible other forms of iron oxides may include FeO, α-Fe₂O₃, β-Fe₂O₃, γ-Fe₂O₃, and ε-Fe₂O₃. For example, a superparamagnetic iron oxide may be used. In one embodiment, maghemite (γ-Fe₂O₃) may be used. A mixture of different iron oxides may also be used. For example, a mixture of magnetite and maghemite may be included in particle 102.

In some embodiments superparamagnetic iron oxide (SPIO) nanoparticles may be used. For example, ultrasmall superparamagnetic iron oxide nanoparticles (USPIO), which have an average individual particle size of about 10 to 40 nm, may be used in some embodiments. The USPIO may be monocrystalline iron oxide nanoparticles (MION) with an average particle size of about 10 to about 30 nm. The SPIO nanoparticles may also have particle sizes from about 60 to about 150 nm, or from about 300 nm to about 3.5 μm, depending on the particular application. Particle 102 may include a single iron oxide crystal, or multiple iron oxide crystals. As can be appreciated by those skilled in the art, single-crystal particles have some properties that are not present in multi-crystal particles, which may conveniently provide certain benefits in some applications.

It is not necessary that particle 102 is entirely formed of a magnetic material. Particle 102 may include other materials that are specifically included for a desired function or materials that are incidentally included during manufacturing or processing. For example, a surface treatment material may be applied to the particle surface to modify, e.g., the solubility of the particle in a given solvent such as water. For instance, particle 102 may include a hydrophilic polymer coating. Particle 102 may also include a component material for labeling or imaging purposes. For instance, an optical label or marker such as a fluorescent material may be included in particle 102. In some embodiments, particle 102 may be an aggregate of two or more smaller individual particles. The different individual particles may be formed of the same material or different materials. For instance, particle 102 may be a heterodimer particle.

Surface modifier 104 is formed of one or more small molecules that have specific binding affinities to selected target cells, and is used to modify the particle surface so that the modified particle can selectively attach to selected target cells, the benefits of which will become apparent below. A small molecule is not a polymer and has a relatively low molecular weight. Typically, small molecules have a molecular weight of less than 800 Da. Small molecules can bind with high affinity to a biopolymer such as protein, nucleic acid, or polysaccharide, and, when attached the biopolymer, may alter the activity or function of the biopolymer. Two or more surface modifying molecules may be linked to each particle 102, as illustrated in FIG. 1. The target cells may be insulin secreting beta cells, hepatocyte cells, neuron cells, or other cells having specific affinity to a small molecule. The surface modifier may be selected so that it has an affinity to a cell surface marker that is not internalized by the cell.

In the exemplary embodiment, surface modifier 104 includes a glucosamine. The surface modifier 104 may be formed from maleimido glucosamine, 2-Amino-2-deoxy-D-glucose hydrochloride, Chitosamine hydrochloride, D-(+)-Glucosamine hydrochloride, N-Acetyl-D-glucosamine, D-Glucosamine 6-sulfate, D-Glucosamine 6-phosphate, or the like. Derivatives or variations of the above listed chemicals may also be used as long as the amine functional group is retained.

A glucosamine can be an efficient surface modifier for specific attachment to certain cells such as insulin-secreting beta cells and for separating such cells from other cells. Without being limited to any particular theory, it is expected that a glucosamine can bind to the glucose transporter Glut2. As Glut2 is specifically expressed in certain cells such as in insulin-secreting beta cells but not in other cells, a glucosamine has specific binding affinity to insulin-secreting beta cells or cells in which Glut2 is expressed. It has been reported in the literature that Glut2 has a higher affinity for glucosamine than for glucose.

As can be appreciated, other similar molecules such as glutamine or galactose also have specific affinity to certain types of cells and may also be used as surface modifiers. However, for attachment to cells which express Glut2 receptors such as beta cells, a glucosamine surface modifier can provide a high attachment efficiency and selectivity, as it has high affinity to Glut2 but low affinity to other cells that do not express Glut2 receptors. In contrast, galactose and glutamine do not have high affinity to beta cells, as their corresponding receptors are not generally expressed in beta cells.

Linker 106 has a protease recognition site and includes a peptide bond, such that cleavage of the peptide bond is catalyzed by a specific protease that recognizes the protease recognition site. In other words, linker 106 includes a cleavable peptide bond specific to a selected protease. The cleavage (breaking up) of a peptide bond specific to a protease will be catalyzed by the specific protease. Linker 106 links particle 102 and modifier 104 through the cleavable peptide bond, and is selected such that when conjugate 100 is exposed to the specific protease, cleavage of the peptide bond is catalyzed to sever the link between particle 102 and modifier 104. The benefits of providing a protease-specific peptide bond in the link will become apparent below.

Suitable molecules for linker 106 include, for example, small molecules having a specific recognition sequence recognized by a selected protease. For example, a primary recognition sequence for thrombin may be expressed as P₄-P₃-Pro-Arg/Lys-cut-P₁′-P₂′[SEQ ID NO: 1] where P₃ and P₄ are hydrophobic and P₁′ and P₂′ are non-acidic. Examples of such recognition sequences include Leu-Val-Pro-Arg-cut-Gly-Ser [SEQ ID NO: 2] (pGEX-T vectors), Met-Tyr-Pro-Arg-cut-Gly-Asn [SEQ ID NO: 3], and Ile-Arg-Pro-Lys-cut-Leu-Lys [SEQ ID NO: 4] (inexact). A secondary recognition sequence for thrombin may be expressed as P₂-Arg/Lys-cut-P₁′, where either P₂ or P₁′ is Gly. For example, a secondary recognition sequence may be Ala-Arg-cut-Gly or Gly-Lys-cut-Ala. In the above expressions, the possible cleavage sites are indicated by ‘cut’; and when a residue can be one of two amino acids a slash (/) is used to separate the two possibilities. In one embodiment, thrombin is the selected protease, and linker 106 comprises a recognition sequence for thrombin, such as a sequence described above. For instance, linker 106 may include the sequence of cys-Leu-Val-Pro-Arg-Gly-Ser-gly-cys-gly [SEQ ID NO: 5].

For serine proteases (includes trypsin), linker 106 may include a recognition sequence of LIVMSTASTAGHC [SEQ ID NO: 6], in which case, the protease cuts at H. For cysteine proteases such as Tobacco Etch Virus (TEV), linker 106 may include a recognition sequence of ENLYFQ(G/S) [SEQ ID NO: 7], in which case, cleavage occurs between the Gln and Gly/Ser residues. In selected embodiments, a linker 106 may be selected so that it is susceptible to a protease that does not adversely impact a function of a cell to which the conjugate is attached. For example, linker 106 my be selected so that it is susceptible to a protease that does not cleave cell surface domains of particular proteins, such as proteins that are required for cellular adhesion or signaling.

In selected embodiments, the protease recognition sequence may be, form, or constitute, a protease recognition site.

As can be understood by those skilled in the art, in some embodiments the protease recognition site may be the site at which cleavage of the linker takes place. However, in other embodiments the protease recognition site may be different from the site at which cleavage of the linker occurs.

Linker 106 should be suitable for attachment to particle 102, either chemically or physically. Linker 106 may include a terminal group that can bind with the surface of particle 102.

Modifier 104 and linker 106 may be chemically bonded, and may be provided in a single molecule. The modifier and the linker may also be attached to one another through physical bonding.

A further exemplary embodiment of the present invention relates to a process for preparing a conjugate such as conjugate 100. While conjugate 100 may be formed according to the processes described herein, it may also be prepared by other processes as will be understood by those skilled in view of present disclosure.

In an exemplary process, particle 102 may be prepared using any suitable technique. For example, suitable techniques for making magnetic particles comprising magnetite are known to those skilled in the art. Exemplary suitable techniques are disclosed in N. R. Jana et al., Chem. Mater., 2004, vol. 16, p. 3931-3935 (referred to herein as “Jana”); J. Park et al., Nat. Mater., 2004, vol. 3, p. 891-895 (referred to herein as “Park”); or M. V. Kovalenko et al., J. Am. Chem. Soc, 2007, vol. 129, p. 6352-6353 (referred to herein as “Kovalenko”), the entire contents of each of which are incorporated herein by reference. A specific example is also described in Example I below. Magnetite nanoparticles with different sizes and shapes may be prepared by changing experimental conditions, such as reaction temperature, and the surfactant type used in the process, and concentrations of different reagents. For instance, spherical particles may be prepared by using oleic acid as the surfactant and cubic particles may be prepared by using sodium oleate as the surfactant. The preparation conditions may be adjusted according to the procedures described in Jana, Park and Kovalenko.

Suitable magnetic particles may also be obtained from various commercial sources. For example, suitable magnetic particles may be obtained from Miltenyi Biotec™, Stemcell Technologies™, Invitrogen™, or the like. The raw materials obtained from a commercial source may be used directly or may be further treated before use.

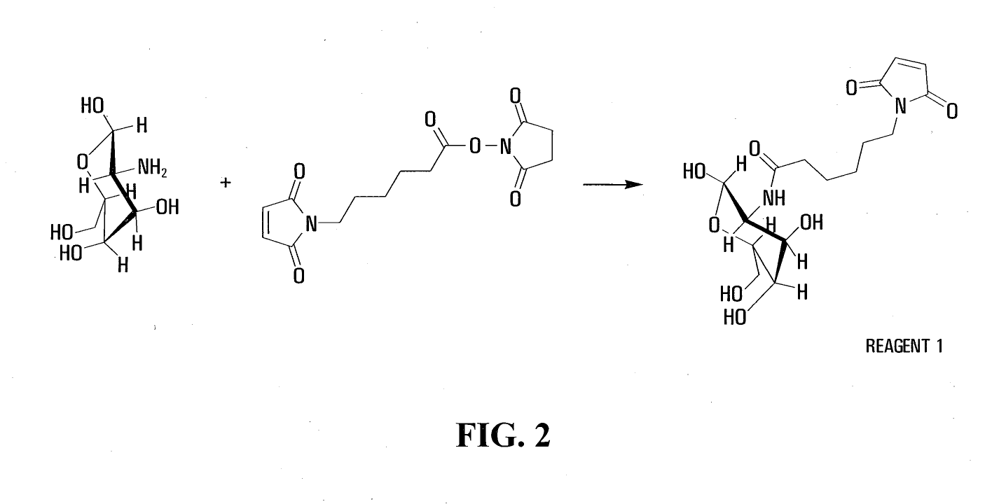

Surface modifier 104 such as a suitable glucosamine may also be prepared by any process known to skilled person in the art for forming glucosamine. Surface modifier 104 or its precursor material may be obtained from commercial sources such as from Sigma Aldrich™, Merck™, or the like. A specific exemplary synthesis route for preparing a suitable modifier is shown in FIG. 2, and described in Example IIA.

Suitable severable linker materials or their precursor materials may be obtained from commercial sources, such as Genescript™. Linker materials may also be prepared according to known techniques for preparing peptide materials.

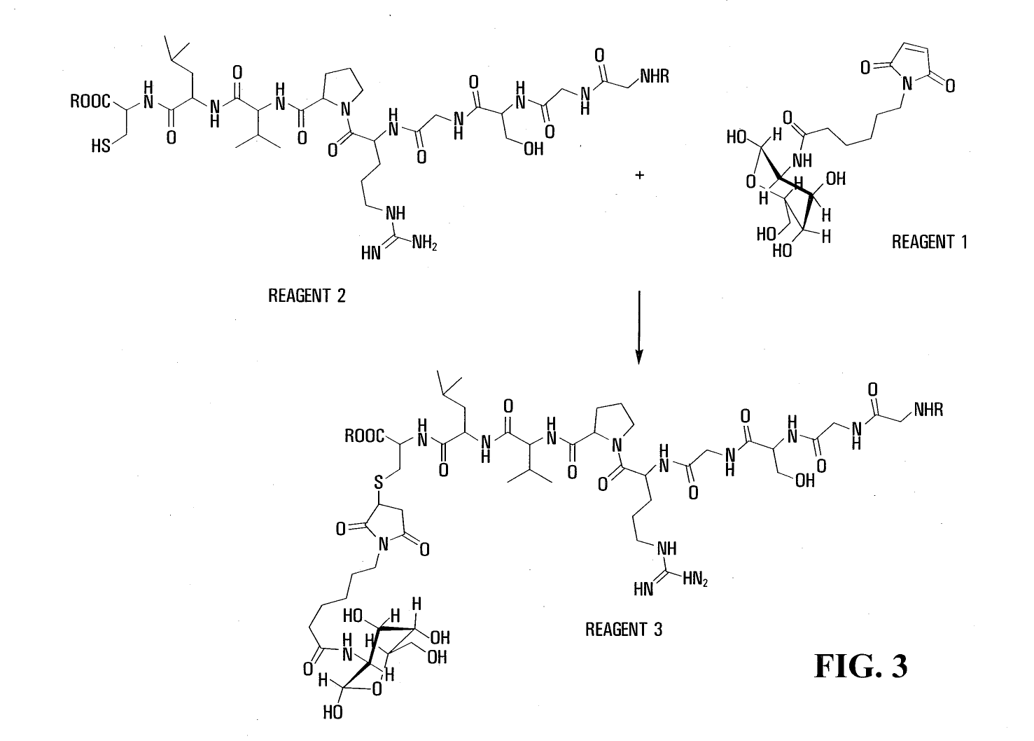

The precursors for modifier 104 and linker 106 may be initially reacted to form a modifier-linker complex. A specific example is shown in FIG. 3, and described in Example IIB. The linker in the modifier-linker complex is then bonded to the surface of particle 102. The procedures for forming the complex and bonding it to the particle will depend on the particular materials used and can be determined by those skilled in the art. Specific exemplary procedures are described in Examples II and III below.

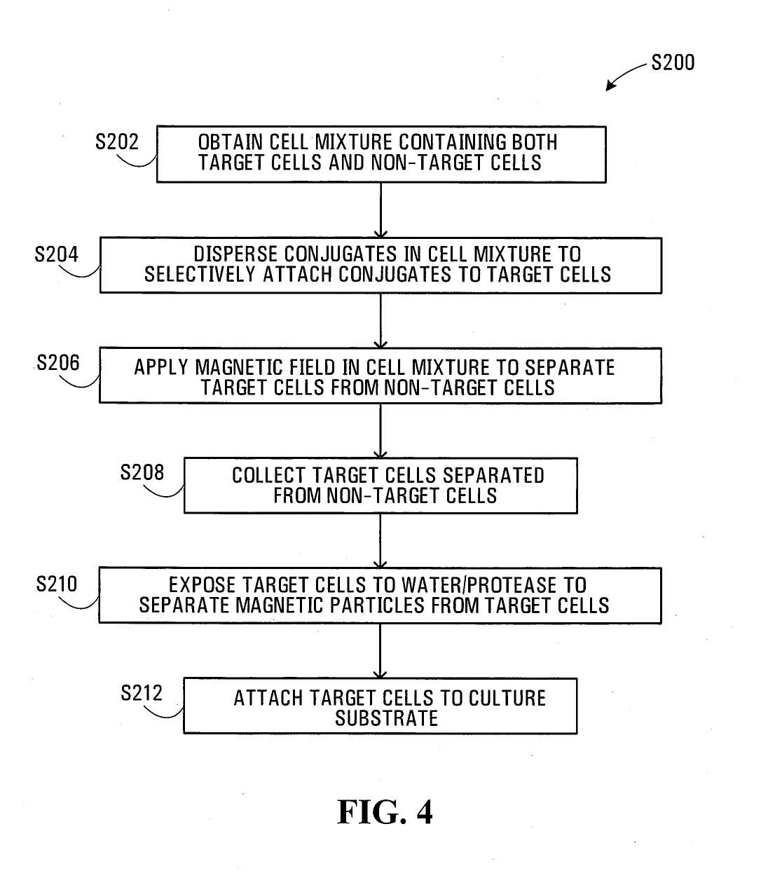

The conjugates described herein can be used to process and manipulate cells. In an exemplary embodiment, conjugate 100 may be used for separating target cells from non-target cells, as illustrated in the process S200 of FIG. 4. As will become apparent, in process S200 and similar procedures involving manipulation of cells, conjugate 100 may be replaced with other conjugates of magnetic particle and surface modifier having specific affinity to the target cell, where the particle and the modifier are linked by a linker that contains a cleavable peptide bond specific to a protease. However, for simplicity of description, conjugate 100 is used below to represent all such conjugates unless otherwise specified. It is also noted that multiple conjugates each having the general structure of conjugate 100 are collectively referred to herein as conjugates 100.

At S202, a mixture of target cells and non-target cells is obtained. Such mixtures are common from normal cell sources in practice. However, it is often desirable to separate the target cells from the non-target cells for various reasons as understood by those skilled in the art. As can be understood, sometimes it is not known if a cell sample obtained from a given source contains a mixture of cell types. Such samples may also be treated according to process S200 to remove potentially present non-target cells. The cell mixture may be provided in a solution such as an aqueous solution so that the cells are free to move about.

At S204, conjugates 100 are dispersed in the cell mixture to allow the conjugates to selectively attach to target cells due to the specific affinity of the surface modifier 104 to the target cells.

Attachment of conjugates 100 to the target cells may be effected by bonding between modifier 104 and a receptor on the cell surface. For example, if Glut2 is expressed in the target cells, and the surface modifiers of the conjugates contain glucosamine, glucosamine can bind with Glut2 in the target cells.

Conjugates 100 are less likely to attach to non-target cells as they have less affinity to bind with the non-target cells, as compared to target cells. As can be appreciated, it is not necessary that all target cells are bonded to conjugates 100 and all non-target cells are not bonded to conjugates 100. As long as more target cells than non-target cells are bonded with conjugates 100, the percentage of target cells in total cells in the cell population can be increased using the process S200 and some benefits can be obtained. Of course, as can be appreciated by those skilled in the art, when the difference in binding affinity of modifier 104 to target cells and non-target cells is larger, the separation efficiency can be increased.

At S206, as conjugates 100 attached to the target cells are magnetic, the target cells may be conveniently manipulated using a magnetic force. For example, a magnetic field may be applied to the cell mixture. The non-target cells that are not bonded with conjugates 100 or another magnetic material will not be subject to the same magnetic force, and as a result, their movement will be different from the movement of the target cells bonded with conjugates 100 under the magnetic field. This effect can be utilized to separate or sort the target cells.

For example, when the cells are suspended in a solution, a magnetic force may be applied to force the target cells to move in a given direction while the non-target cells stay in place.

In another example, a magnetic force may be applied to hold the target cells in place and a fluid flow may be used to flush out the non-target cells.

In some embodiments, cell separation may be effected with the use of a magnetic column as illustrated in the Examples, and as can be understood by those skilled in the art. For instance, the cells may be separated using the magnetic-activated cell sorting (MACS) technique known to persons skilled in the art. Cell separation and purification may also be effected using a flow cytometry technique, which is also known to persons skilled in the art.

Other techniques for cell separation with a magnetic force may also be used as understood by those skilled in the art.

At S208, the separated target cells are collected. The collected cell population will have a higher purity of target cells as compared to the original cell mixture.

Either before or after S208, target cells may also be conveniently subject to other types of magnetic processing. Magnetic processing may include any process that utilizes the magnetic properties of the magnetic particles attached to the target cells. Exemplary magnetic processing includes magnetic detection, magnetic imaging, manipulation with magnetic force, or the like. For example, superparamagnetic iron oxide nanoparticles are expected to be good T2 contrast-enhancing agents, if the conjugates contain magnetite nanoparticles, the target cells may be conveniently studied or analyzed using a magnetic resonance imaging (MRI) technique.

At S210, the target cells are exposed to water and the specific protease that will catalyze cleavage of the peptide bond in the linker 106. For example, for linkers containing glucosamine, the protease may be thrombin as the peptide bonds in glucosamine are specific to thrombin.

As can be understood by those skilled in art, peptide bonds can be cleaved, or broken, by amide hydrolysis in the presence of water. Amide hydrolysis of peptide bonds may occur spontaneously but the reaction is very slow in normal conditions and in the absence of an enzyme that catalyzes the hydrolysis reaction.

When the cleavage of the peptide bond is catalyzed by the protease, severance of the link between the magnetic particle and the target cell can occur within a practical period of time, such as from about to 15 to 60 minutes, or within about 30 minutes.

As can be appreciated, when peptide bonds specific to a protease are used in linker 106, severance of the linker can be conveniently controlled. When the specific protease is not present, cleavage of linker 106 is unlikely to occur quickly under normal conditions even if water is present. Thus, the magnetic particles can remain attached to the target cells for extended periods of time and during magnetic processing if conjugates 100 are not exposed to the specific protease. The specific protease can be mixed with the target cells attached to conjugates 100 in an aqueous environment such as an aqueous solution, when it is the desired time to sever the link between the magnetic particles and the target cells.

Severance of the link can be confirmed, for example, by applying a magnetic field to the cell population and observing the movement of the target cells. If the movement of the target cells is unaffected by the applied field, it indicates that the link with the magnetic particles has been severed.

The target cells released from the magnetic particles can be collected under a magnetic field, as the released cells will move differently from those cells that are still attached to magnetic particles in the magnetic field.

At S212, the released target cells are attached to a culture substrate. This attachment may be effected using any suitable techniques known to those skilled in the art. As the target cells are no longer bonded to magnetic particles 102, interference from such particles can be conveniently avoided. A culture substrate can be any supporting structure on which cells can be cultured. For example a culture substrate may be a culture plate, a culture flask, or the like.

As now can be appreciated, a conjugate of a magnetic particle and a surface modifier having a specific affinity to selected target cells can conveniently be used in processing of cells when the particle and modifier are linked through a cleavable peptide bond specific to a selected protease. While specific exemplary conjugates are described for illustration purposes herein, in different applications variations and modifications of the specifically disclosed examples may be possible, as can be understood by those skilled in the art. For example, different magnetic particles or different surface modifiers may be used in the conjugates. The linker linking the modifier to the magnetic particle may have a different structure and may include additional components, as long as cleavage of the peptide bond will sever the link between the particle and the modifier, and cleavage of the peptide bond can be catalyzed by exposing the conjugate to the specific protease.

Conveniently, by selecting surface modifier that has higher specific affinity to the target cells, cell processing efficiency and effectiveness may be improved.

Also conveniently, a conjugate disclosed herein may be cleaved to facilitate subsequent cellular processes, such as cellular adhesion.

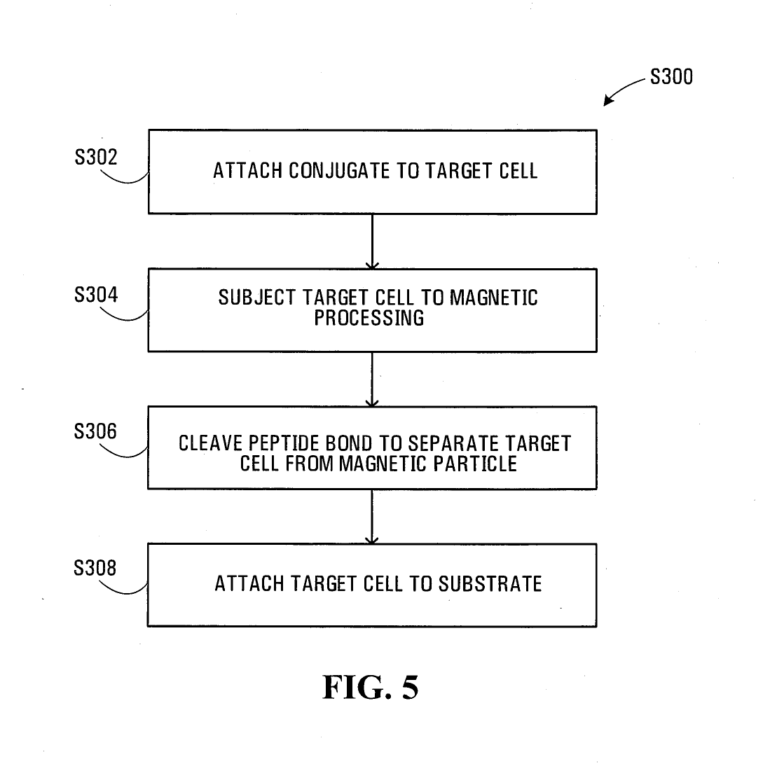

In an exemplary embodiment, cell processing may be performed as illustrated in the process S300 of FIG. 5. At S302, a conjugate is attached to a target cell. The conjugate has a magnetic particle and a surface modifier selected to have a specific binding affinity to the target cell. The particle and modifier are linked through a cleavable peptide bond. The target cell attached to the conjugate is then subject to magnetic processing at S304. After magnetic processing, the peptide bond is cleaved to separate the target cell from the magnetic particle at S306. The target cell separated from the magnetic particle can then be conveniently attached to a substrate at S308. The conjugate may be conjugate 100. The peptide bond may be selected such that cleavage of the peptide bond is catalyzed by a specific protease, such as thrombin. Thus, severance of the link between the magnetic particle and the cell may be effected by exposing the peptide bond to the specific protease. In this embodiment, the surface modifier may be a glucosamine, glutamine, galactose, or another small molecule that has specific affinity to a given type of target cells. The magnetic particle may be a quantum dot or a nanoparticle. For example, magnetite nanoparticles may be used. The linker should be suitable for attachment to the magnetic particle, and may include a terminal group that can bind with the surface of the magnetic particle either by a chemical bond or by physical bonding. The modifier and the linker may be chemically bonded, and may be provided in a single molecule. The modifier and the linker may also be attached to one another through physical bonding.

In another exemplary embodiment, a conjugate for attachment to a cell is formed by linking a surface modifier to a magnetic particle through a cleavable peptide bond. The surface modifier is selected to have a specific affinity to the cell. The peptide bond is selected such that cleavage of the peptide bond is catalyzed by a specific protease, so that cleavage of the peptide bond can be conveniently effected by exposing the conjugate to the specific protease. In this embodiment, the protease may be thrombin. The surface modifier may be a glucosamine, glutamine, galactose, or another small molecule that has specific affinity to the cell. The magnetic particle may be a quantum dot or a nanoparticle.

Suitable surface modifiers may be small molecules with a functional group that has different binding affinities to surface receptors on different types of cells. A larger difference in the binding affinities to target cells and non-target cells may provide more selective attachment to the cells, and thus increased processing efficiency.

The target cells may be any cells that have surface receptors for specifically binding with the selected surface modifier. For example, with a glucosamine as the surface modifier, insulin-secreting beta cells may be the target cells as the glucosamine modifier has high binding affinity to the Glut2 receptors on the cell surface. It has been found that insulin-secreting beta cells attached with conjugates of magnetite nanoparticle and glucosamine can be effectively separated from surrounding (non-target) cells by applying a magnetic field to the cell population. The cell population can thus be purified, for example, to have up to 80% of insulin-secreting beta cells.

In at least some embodiments, when the exemplary conjugates are used in cell processing, the linker in the conjugates, such as linker 106, should be selected so that the corresponding specific protease will not adversely impact the viability of the cells when it is used to cleave the linker, including not interfering with a subsequent attachment of the cell to a substrate. Accordingly, the protease should be selected so that it does not recognize the surface proteins on the cells, or at least the important surface protein(s), such as a protein involved in the subsequent substrate attachment process. In other words, the recognition sequence for the protease should not be present on the surface of the target cells and other useful cells in the cell mixture.

More generally, it should be understood that when used with cells, the conjugates, particularly their surface materials and any portions of the conjugates that may interact with the attached or surrounding cells, should be formed with materials that are biocompatible with the cells and will not have significant adverse effects such as toxic effects on the cells.

The conjugates disclosed herein can find use in many different cell processing applications. For instance, as discussed above, the conjugates can be used in cell separation applications. As cell separation is a common step in many biomedical and tissue engineering applications based on cells, embodiments of the present invention are useful in such biomedical applications.

Cell separation may be used to remove unwanted cells, which may trigger the malfunction of the specified cells of interest. For example, the presence of unwanted myoblasts or other cell types in a cardiomyocyte population may hinder the synchronous beating behavior of cardiomyocytes. In another example, unwanted kidney tubule epithelial cells would transform into fibrotic cells when cultured along with fibroblasts.

Using the embodiments disclosed herein, insulin-secreting beta cells may be conveniently separated, for example, from embryonic stem cells (ESCs) such as after differentiation therefrom, from induced pluripotent cells (iPS), or from adult stem cells such as bone marrow mesenchymal stem cells (MSCs).

The conjugates disclosed herein can also be used in applications utilizing a chromatography technique, such as a column chromatography technique. An exemplary column chromatography technique is the expanded bed absorption (EBA) technique.

Other applications and uses of the conjugates are also possible as can be understood by those skilled in the art.

Exemplary embodiments of the present invention are further illustrated with the following examples, which are not intended to be limiting.

### EXAMPLES

### Example I

### Synthesis of Sample Iron Oxide Nanoparticles

Iron oxide nanoparticles were synthesized by thermal decomposition of iron-oleate as described in Jana N. R., et al., “Size- and shape-controlled magnetic (Cr, Mn, Fe, Co, Ni) oxide nanocrystals via a simple and general approach,” Chem. Mater. (2004), vol. 16, pp. 3931-3935; and Park J., et al., “Ultra-large-scale syntheses of monodisperse nanocrystals,” Nat. Mater. (2004), vol. 3, p. 891, the entire contents of each of which are incorporated herein by reference. Briefly, anhydrous FeCl3 (1.63 g, 10 mmol) and sodium oleate (9.125 g, 30 mmol) were added to a mixture of ethanol (20 ml), deionized water (20 ml) and hexane (30 ml). The mixture was refluxed at 70° C. for 4 h. The reddish brown solution containing the iron-oleate complex was washed three times with deionized water in a separation funnel. Hexane was evaporated using a rotary evaporator, yielding an oily iron-oleate complex.

The iron oleate complex was dissolved in 1-octadecene (25 g), and oleic acid (1.41 g, 5 mmol) or sodium oleate (1.52 g, 5 mM) was next added. The mixture was heated to 320° C. and maintained at that temperature for 1 h. The resulting black solution was cooled to room temperature and 2-propanol was next added to precipitate the magnetic particles. The particles were further centrifuged and washed with hexane and ethanol, and redispersed in hexane or toluene. The resulting iron oxide nanoparticles were used as the sample iron oxide nanoparticles in other examples described herein, and are referred to as Sample I.

### Example II

### Synthesis of Peptide-Glucosamine

### IIA. Conjugation of Maleimide to Glucosamine

The basic reaction for this synthesis procedure was as shown in FIG. 2. A flame dried 5-mL reaction vial was charged with an aqueous stock solution of glucosamine hydrochloride (5 mg in 0.25 mL, 0.021 mmol) and a dry dimethylformamide (DMF, 0.25 mL) stock solution of 6-maleimidohexanoic acid N-hydroxysuccinimide ester (7 mg, 0.022 mmol) under argon atmosphere, and cooled in an ice bath at 0° C. Dry DMF (1 mL) was added dropwise, and the pH of the reaction was adjusted to 8 by carbonate buffer. The reaction mixture was stirred at 0° C. for 2 h under argon, and then brought to room temperature and stirred for another 24 h under argon. DMF was removed under reduced pressure, and the residue was dried under high vacuum to obtain a white residue, which was referred to as Reagent 1 and was used directly in step IIB without further purification.

### IIB. Conjugation of Peptide to Maleimidoglucosamine

The basic reaction for the conjugation process was as shown in FIG. 3. A peptide (Reagent 2 as shown in FIG. 3) (17 mg, 0.02 mmol) was dissolved in phosphate buffer (2 mL, pH 7.2), and was treated with maleimidoglucosamine (Reagent 1). Reagent 2 includes the protease recognition sequence of cys-Leu-Val-Pro-Arg-Gly-Ser-gly-cys-gly. The reaction mixture was covered with an aluminum foil, and stirred under argon for 24 h. The solution was purified by reverse phase recycling high-performance liquid chromatography (HPLC) using a refractive index (RI) detector, and freeze dried to obtain a white powder (20 mg, 82%) product, referred to as Reagent 3 as shown in FIG. 3.

### Example III

### Conjugation of Glucosamine-Peptide Complex to Iron Oxide Particles

15 mg of O,O′-bis[2-(N-succinimidyl-succinylamino)ethyl]polyethylene glycol (biNHS-PEG), a homobifunctional amine reactive crosslinker, was dissolved in 100 μL of dimethylsulfoxide. This was added to the sample iron oxide particles as produced in Example I. The mixture was sonicated for 30 min. Excess PEG linker was added to ensure that there were unreacted NHS groups on the particle surface available for glucosamine conjugation in the next step. The activated nanoparticles were then passed through a PD-10 desalting column rinsed with 10 mM 4-(2-hydroxyethyl)-1-piperazineethanesulfonic acid (HEPES) buffer. The particles were collected and split into 2 separate vials.

Vial 1: 1.5 μmol of the sample glucosamine-peptide (Reagent 3) was dissolved in 1 ml of 10 mM HEPES buffer. This was mixed with the activated iron oxide particles immediately and stirred overnight at 4° C.

Vial 2: 1.5 μmol of glucosamine was dissolved in 1 ml of 10 mM HEPES buffer. This was mixed with the activated iron oxide particles immediately and stirred overnight at 4° C.

The conjugated nanoparticles were centrifuged, and washed with 10 mM of HEPES using a microcentrifuge filter (molecular weight cutoff (MWCO)=30 kDa)). The sample particles collected were used in the following Examples.

Sample particles produced from Vial 1 are referred to as Sample IIIA and sample particles produced from Vial 2 are referred to as Sample IIIB herein.

### Example IV

### Synthesis of Glucosamine-Coated Iron Oxide Nanoparticles (Comparison)

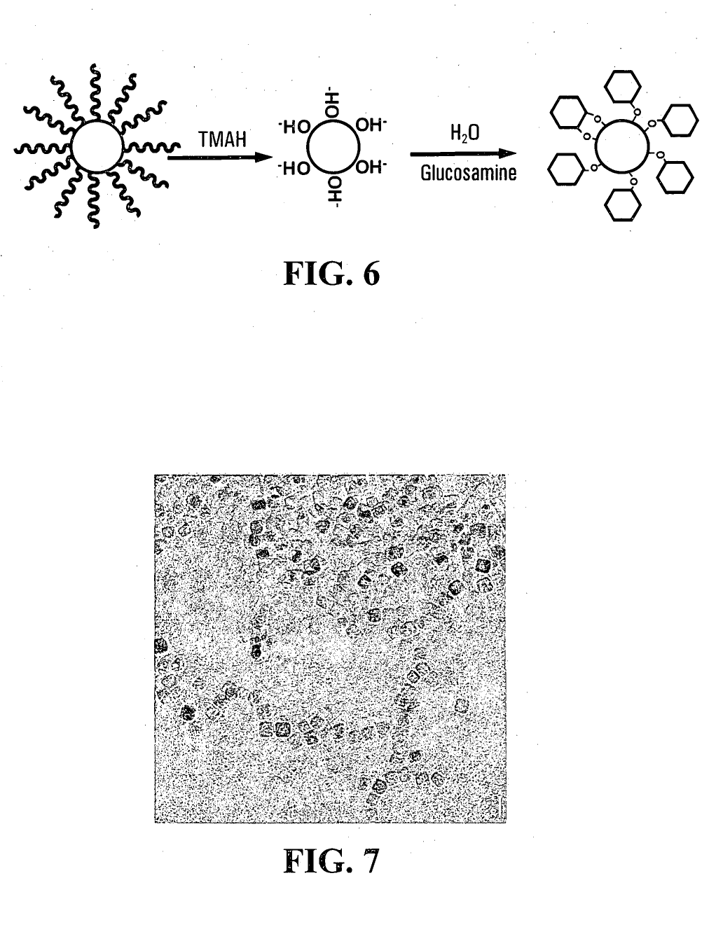

Glucosamine was conjugated to sample iron oxide particles in two steps. The synthesis route is illustrated in FIG. 6. First, sample iron oxide nanoparticles were made hydrophobic via tetramethylammonium hydroxide (TMAH). Next, glucosamine was coated on the surface of the sample particles. Briefly, 1 mg of iron oxide nanoparticles were precipitated and centrifuged by adding an equal volume ratio of ethanol. 0.5 mL of 1 M TMAH in H₂O was then added to the black precipitate, and the mixture was sonicated for 5-10 min. The mixture was left to stand for another 10 min, and then 0.5 mL of acetone was added to precipitate the particles. The particles were then redispersed in deionized water, and washed with acetone.

To coat with glucosamine, sample nanoparticles dispersed in water (1 mg in 250 μL) were added to 1 mg of glucosamine in 2 mL of H₂O. The solution remained clear, and was mixed overnight. The solution was next centrifuged at 25000 g for 30 min, and the particles were collected and redispersed in water. This was repeated once, followed by redispersion in water. The resulting particles remained stable in deionized water for weeks, and will be referred to as Sample IV herein.

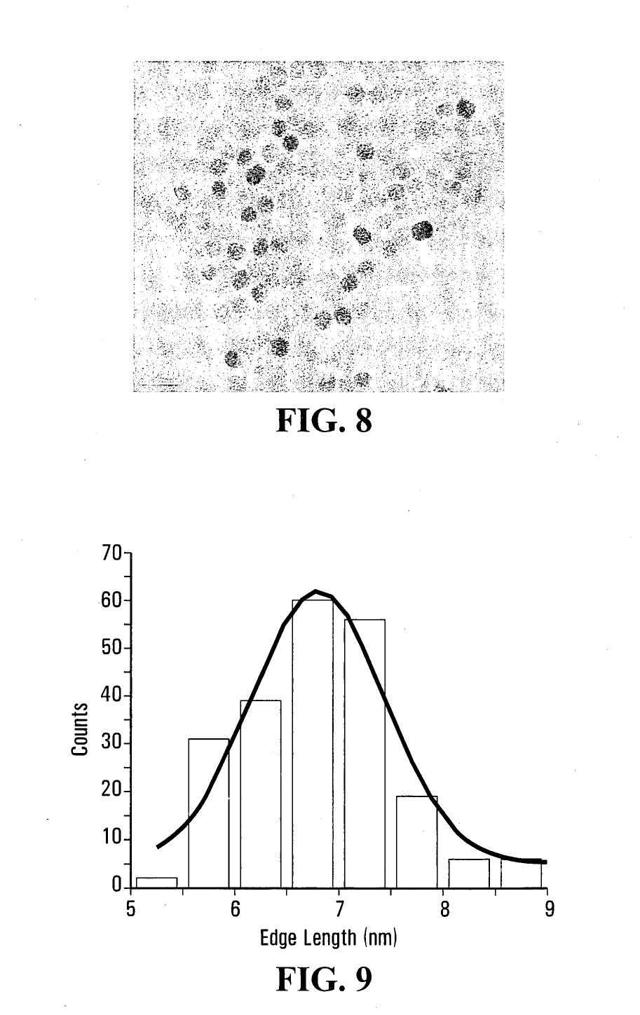

FIG. 7 shows a representative transmission electron microscopy (TEM) image of Sample IV. FIG. 8 shows a representative TEM image of sample iron oxide (magnetite) nanoparticles. FIG. 9 shows dynamic light scattering (DLS) results measured from the sample nanoparticles (the total number of counts was 282, and the average edge length was 6.8±0.6 nm).

### Example V

### Conjugation of D-glucosamine with cGSH-ZnS-CdS-CdSe QDs (Comparison)

1 ml of crosslinked glutathione-capped ZnS-CdS-CdSe (cGSH-ZnS-CdS-CdSe) quantum dot (referred to as “QD⁵⁹⁵”) solution (1 mg/ml) was diluted to 20 ml with 100 mM borate buffer (pH 8.0). 10 mg of N-hydroxysuccinimide (NHS) and 20 mg of 1-ethyl-3-[3-dimethylaminopropyl]carbodiimide hydrochloride (EDC) were freshly dissolved in 2 ml of 100 mM borate buffer, and were immediately added to the QD⁵⁹⁵ solution with stirring. 1 ml of D-glucosamine dissolved in 100 mM borate buffer to a concentration of 1 mg/ml was added. After incubation overnight, the system was quenched with a 50 mM glycine buffer (pH 7.5). Glucosamine-conjugated QDs were purified by ultrafiltration with a membrane of 50 KDa molecular weight cutoff (MWCO).

The resulting glucosamine-conjugated QDs will be referred to as Sample V.

### Example VI

### Attachment of Sample Conjugates to Cells

Sample V conjugates were mixed with insulin-secreting beta cells to attach the conjugates to the cells, by rocking the mixture in a rocker at a speed of 30 rpm/min at 37° C. (5% CO₂).

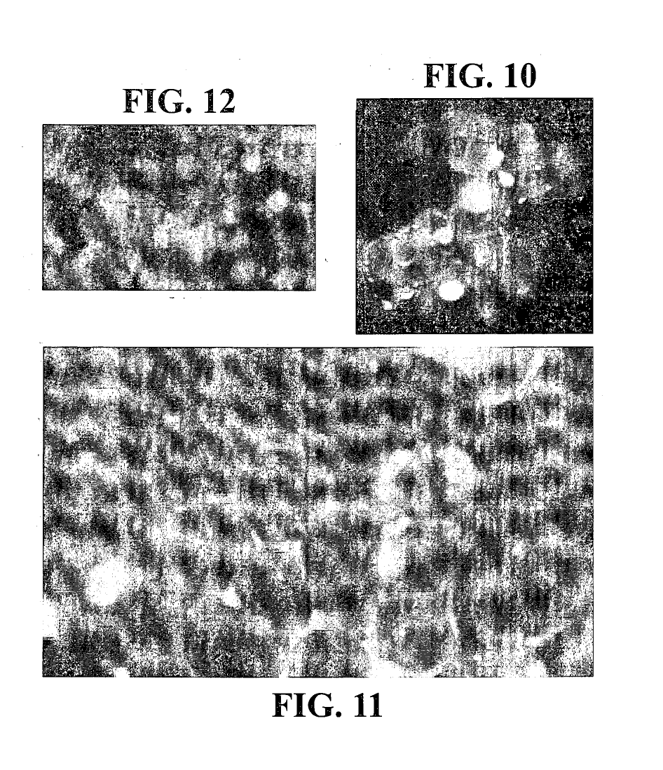

Fluorescence micrographs of the test samples showed a strong presence of the glucosamine-QDs⁵⁹⁵ (λₑₘ=595 nm) on the surface of insulin-secreting beta cells. Representative confocal microscopic images of the tested samples are shown in FIGS. 10 and 11.

For comparison, QDs⁵⁹⁵ without glucosamine were also mixed with insulin-secreting beta cells. It was observed that uptake of the QDs without glucosamine by the cells was non-specific. A representative confocal microscopic images of the tested sample is shown in FIG. 12.

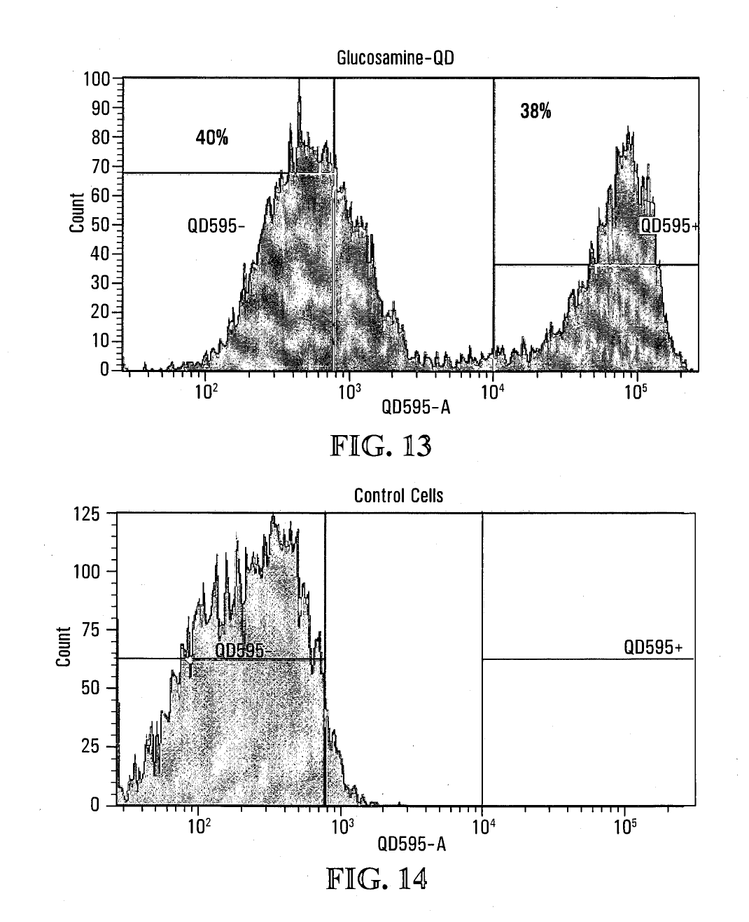

Flow cytometry results indicated that 38% of the cells were labeled as “QD-positive” when Sample V was used FIG. 13 shows the QD uptake distribution for Sample V in a mixture of fibroblasts and insulin-secreting beta cells incubated with Sample V, as analyzed by flow cytometry showing auto-fluorescence.

In comparison, QD update was substantially negative when QDs without glucosamine was used, as can be seen in FIG. 14 which was for the control mixture of fibroblasts and insulin-secreting beta cells incubated with bare QDs.

The positive and negative fractions from the flow cytometry for Sample V were further analyzed for specific genes using real-time polymerase chain reaction (RT-PCR) with gene-specific primers. The fibroblast used contained neomycin gene incorporated in its genome. Hence, the specific markers for these fibroblasts were neomycin and CD90. In comparison, the specific gene targets for insulin-secreting beta cells were insulin and Glut2.

The sample cells were subject to ribonucleic acid (RNA) isolation and two-step RT-PCR as follows. The total RNA was isolated from the cells using the Genelute RNA isolation kit (Sigma™, USA) according to the manufacturer's protocol. 3 μg of DNase I (Rnase free, Invitrogen™) treated total RNA was reverse transcribed into complementary deoxyribonucleic acid (cDNA) with Superscript III (Invitrogen, USA) for 90 min at 42° C. PCR was performed with Advantage 2 Taq polymerase (BD biosciences™, USA). Gene-specific primers were designed from the available sequences from the Singapore National Center for Biotechnology Information gene databank. RT-PCR was conducted in Bio-Rad iCycler™ using TaqMan assay for the specific genes obtained from Applied Biosystems™, USA.

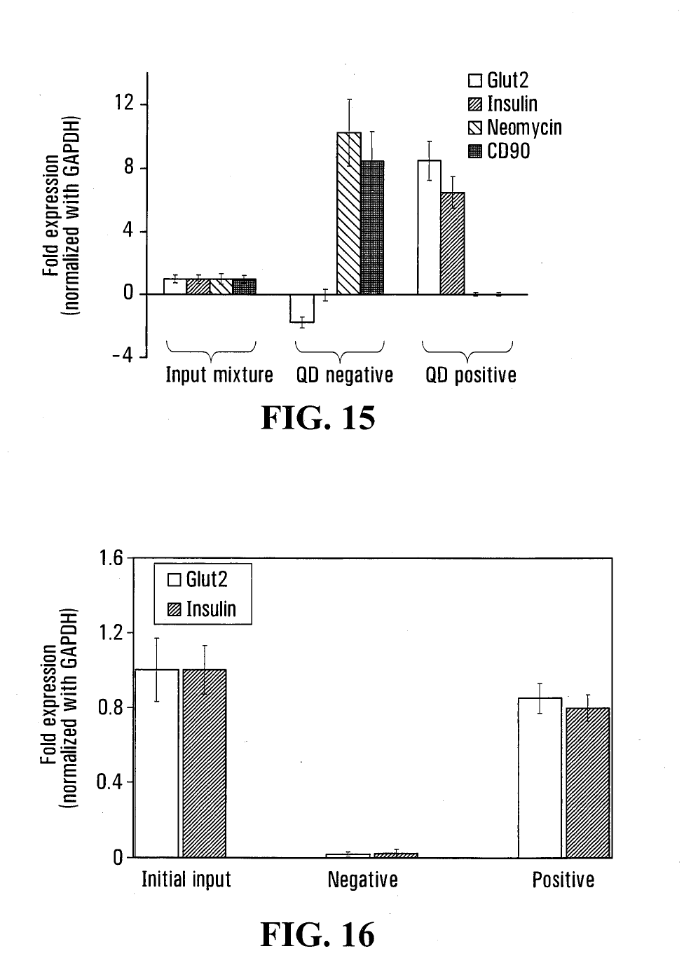

Real-time PCR results indicated that the “QD-negative” fraction and “QD-positive” fraction had strong expressions of the markers associated with fibroblasts and insulin-secreting beta cells, respectively. FIG. 15 shows the representative PCR results, where the gene expression in the initial mixture was used for normalization (i.e. 1-fold).

Separate tests for cell attachments were also performed with Sample IIIA, Sample IIIB, and Sample IV as the respective conjugates.

Test results showed that conjugates of glucosamine and iron oxide nanoparticles exhibited high binding efficiency to insulin cells, and provided up to 80 to 85% of insulin cells recovery in a magnetic column based cell separation process.

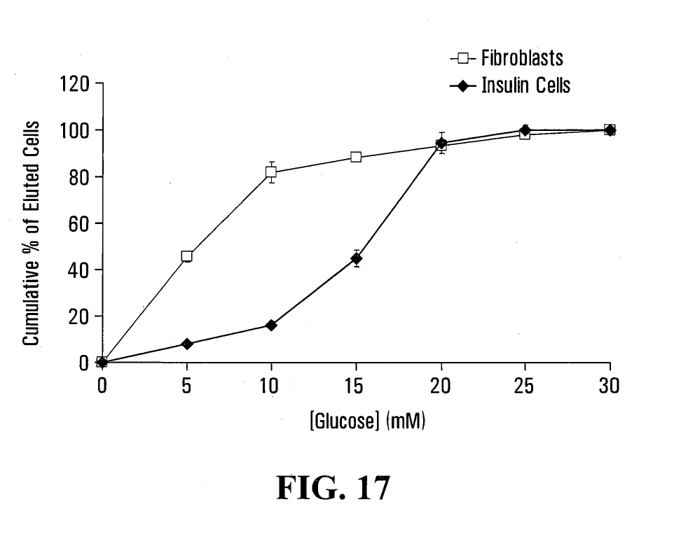

Glucosamine's affinity to Glut2 receptors was tested by eluting glucosamine-bound fibroblasts and insulin cells with different concentrations of glucose. The elution profiles of fibroblasts and insulin-secreting beta cells were different, as shown in FIGS. 16 and 17. FIG. 16 shows the results of real-time PCR analysis of the cells separated using Sample IV conjugates. The gene expressions in the initial cell input was used for normalization (i.e. 1-fold). The flow-through (negative) fraction and the bound (positive) fraction were analyzed for the insulin-secreting beta cell specific gene expression using gene specific primers. FIG. 17 shows the cumulative elution profiles of fibroblasts and insulin cells incubated with Sample IV conjugates under different glucose concentration. It indicated that the binding affinities of fibroblasts and insulin cells to Sample IV conjugates were different. Fibroblasts could be eluted at a lower concentration of glucose (10 mM), while insulin-secreting beta cells required a higher concentration of glucose (20 mM). This result indicated that insulin-secreting beta cells had a higher affinity to glucosamine, as compared to fibroblasts. Glut2 was expressed on insulin-secreting beta cells but not on fibroblasts, which bonded to glucosamine through Glut1. It can thus be expected that Glut2 has a high affinity to glucosamine.

### Example VII

### Cell Separation Tests

Rat insulin-secreting beta cell line (bTC3) was obtained from ATCC™, and neomycin-resistant mouse embryonic fibroblasts were obtained from Millipore. Both cells were cultured in Dulbecco's Modified Eagle's Medium (DMEM) containing 10% fetal bovine serum (FBS) and 1% penicillin-streptomycin.

The cells were dispersed to separate individual cells by adding trypsin. The separated cells were washed with phosphate buffered saline (PBS) (twice) and incubated with Sample IV conjugates produced in Example IV for 1 h in the binding buffer, which was formed of 2% of bovine serum albumin (BSA) and 1 mM ethylenediaminetetraacetic acid (EDTA) in PBS. The cells were passed through a magnetic column attached to a magnet. The column was washed with washing buffer (PBS containing 2% of BSA). The flow-through solution was collected as the negative binding fraction, while the bound fraction was collected upon removal of the magnetic force.

In separate tests, cells labeled with Sample V conjugates (QD⁵⁹⁵) or cytotracker were suspended in PBS containing 5% FBS. The artificially mixed populations of insulin cells (50%) and fibroblasts (50%) were used to test cell separation in a flow cytometry platform with Sample V conjugates.

Samples collected at different stages of cell separations were analyzed using a 3-laser LSR II FACS™ analyzer from BD Biosciences, USA.

Separate tests for cell separation were also performed with Sample IIIA as the attached conjugates.

Using fluorescently labeled fibroblasts and unlabeled insulin-secreting beta cells in cell separation tests, the selective attachment properties of the glucosamine conjugates were verified by flow cytometry. Upon binding of the cells to the magnetic column, the cells were washed with 10 mM glucose (to first remove most of the weakly bound fibroblasts), followed by the elution of the remaining cells bound to the column.

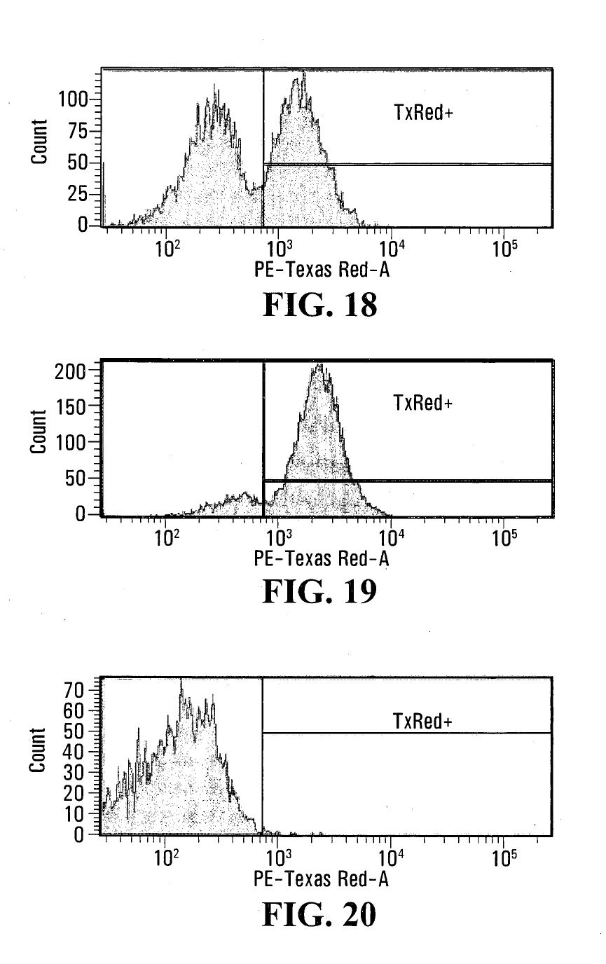

FIG. 18 shows the profiles of the cytometry analysis of the sample mixture of cells prior to separation. The mixture of cells contained mouse fibroblasts labeled with red fluorescence artificially mixed with insulin-secreting beta cells. Insulin-secreting beta cells were separated using Sample IV conjugates. FIG. 19 shows the results of cytometry analysis of the flow-through fraction of the cells that passed the magnetic column after cell separation, which, as can be seen, contained mostly fibroblasts (˜85%). FIG. 20 shows the results of cytometry analysis of the bound fraction of cells after cell separation, which contained mostly insulin-secreting beta cells (˜75%). The flow cytometry results indicated that 85% of the fibroblasts were recovered in the 10 mM glucose wash fraction, and that the bound fraction contained mainly (˜75%) unlabeled cells (insulin-secreting beta cells).

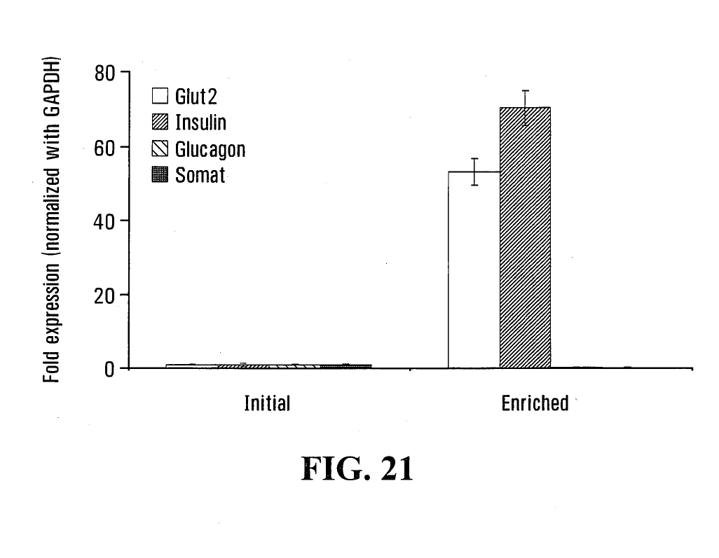

Tests were also conducted to enrich insulin-secreting beta cells from whole pancreas of pigs. Pancreatic islets contained mainly 3 types of cells, alpha cells (˜15% of islet cells, identified by glucagon expression), insulin-secreting beta cells (˜80% of islet cells, identified by insulin), and Glut2 and delta cells (˜3% of islet cells, identified by somatostatin expression). Islets were isolated from the pig pancreas and treated with collagenase to form single cells. These cells were incubated with Sample IV conjugates. The conjugate-bonded cell fraction (“enriched”) was analyzed for gene expression by real-time PCR. The results are shown in FIG. 21. The enriched fraction was found to have strong expressions of the markers associated with beta cells. The real-time PCR results showed that the enriched population contained mainly the insulin- and Glut2-expressing insulin-secreting beta cells. Furthermore, the absence of expression of somatostatin and glucagon confirmed that the enriched insulin-secreting beta cell population was not contaminated by the surrounding islet cells, such as alpha cells and delta cells.

### Example VIII

### Cleavage of Links Between Cells and Magnetic Particles

Tests were conducted to confirm that the links between iron oxide nanoparticles and the cells could be cleaved by exposure to thrombin. In these tests, sample cells bonded to iron oxide particles by way of Sample IIIA conjugates were incubated with 50 units of thrombin (total volume=0.5 ml) at 37° C. for 30 min. The suspension was then exposed to magnetic field and the unbonded fraction was collected.

### Example VIII

### Attachment of Cells to Substrate

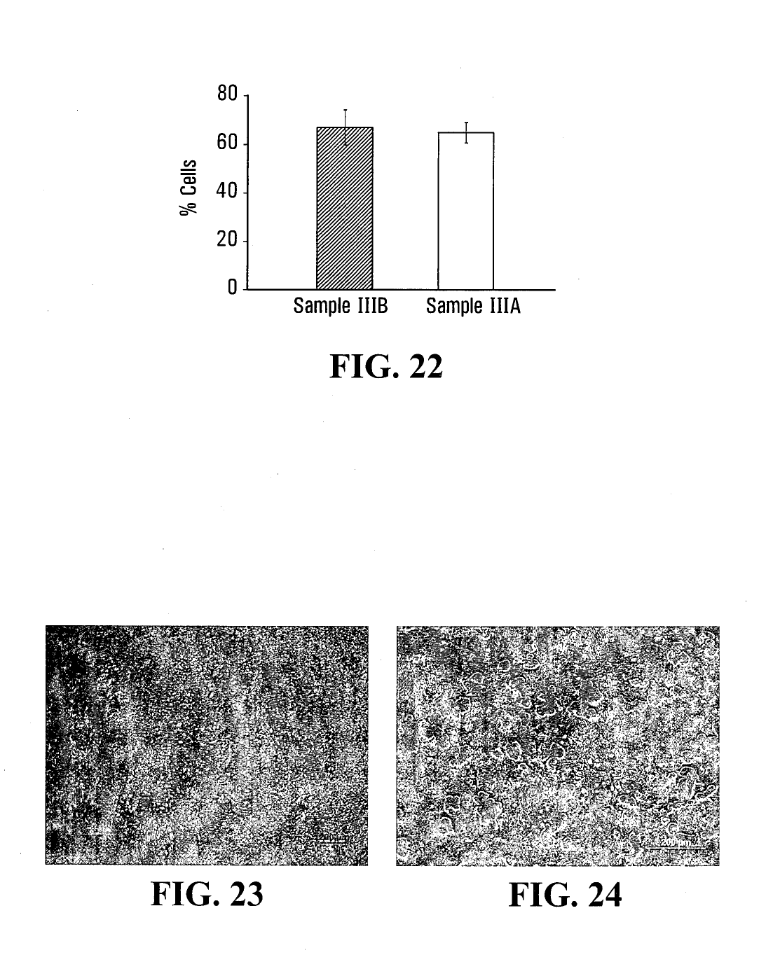

Insulin cells were incubated with Sample IIIA and IIIB conjugates respectively. Sample IIIA conjugates contained a thrombin-specific peptide linking glucosamine to the iron oxide particle. Sample IIIB conjugates did not contain a peptide linker. The cells attached with the conjugates were subject to magnetic field separation and collected. As shown in FIG. 22, the percentage of cells attached with the conjugates was similar for both Samples IIIA and IIIB.

The collected cells bonded to Sample IIIB were cultured directly on tissue culture plates (substrate).

The collected cells bonded to Sample IIIA were incubated with 50 units of thrombin for 30 min at 37° C. as described in Example VII, and then subject to further magnetic field separation. The flow-through fraction that contained the released cells was collected, and cultured on tissue culture plates (substrate).

Representative images of the respective culture plates taken after 24 h of culturing are shown in FIGS. 23 (for Sample IIIB) and 24 (for Sample IIIA), respectively. It was observed that the separated insulin cells attached to Sample IIIB failed to adhere and proliferate, like the control cells that were not subject to the cell separation procedure. It was also observed that the cells released from the magnetic particles in Sample IIIA conjugates successfully adhered to the culture substrate.

As used herein, and unless otherwise specifically indicated to the contrary, the term “comprise”, including any variation thereof, is intended to be open-ended and means “include, but not limited to.”

When a list of items is given herein with an “or” before the last item, any of the listed items or any suitable combination of the listed items may be selected and used. For any list of possible elements or features provided in this specification, any sublist falling within a given list is also intended. Similarly, for any range provided, any subrange falling within a given range is also intended.

Of course, the above described embodiments are intended to be illustrative only and in no way limiting. The described embodiments are susceptible to many modifications of form, arrangement of parts, details and order of operation. The invention, rather, is intended to encompass all such modification within its scope, as defined by the claims.

1. A conjugate comprising:
a magnetic particle comprising an iron oxide;
a surface modifier comprising a glucosamine; and
a linker comprising a protease recognition site and a peptide bond, wherein said linker links said surface modifier to said particle, and wherein cleavage of said peptide bond is catalyzed by a specific protease that recognizes said protease recognition site.

2. The conjugate of claim 1, wherein said protease is thrombin.

3. The conjugate of claim 1, wherein said particle comprises a quantum dot.

4. The conjugate of claim 1, wherein said particle is a nanoparticle.

5. The conjugate of claim 1, wherein said particle is superparamagnetic.

6. The conjugate of claim 1, wherein said particle comprises magnetite.

7. The conjugate of claim 1, wherein said linker comprises a protease recognition sequence.

8. The conjugate of claim 7, wherein said protease recognition sequence comprises Leu-Val-Pro-Arg-Gly-Ser.

9. A method of cell processing, comprising:
attaching a conjugate to a target cell, said conjugate comprising a magnetic particle, a surface modifier having a specific affinity to said target cell, wherein said particle and modifier are linked through a cleavable peptide bond;
subjecting said target cell attached to said conjugate to magnetic processing;
cleaving said peptide bond to separate said target cell from said magnetic particle; and
providing a substrate and allowing said target cell separated from said magnetic particle to attach to said substrate.

10. The method of claim 9, wherein said conjugate comprises a linker linking said surface modifier to said magnetic particle, said linker comprising a protease recognition site and said peptide bond, wherein cleavage of said peptide bond is catalyzed by a specific protease that recognizes said protease recognition site, and wherein said cleaving comprises exposing said linker to said protease.

11. The method of claim 10, wherein said protease is thrombin.

12. The method of claim 9, wherein said surface modifier comprises a glucosamine, glutamine, or galactose.

13. The method of claim 9, wherein said magnetic particle comprises a quantum dot or a nanoparticle.

14. The method of claim 9, wherein said magnetic particle is superparamagnetic.

15. A method of cell processing, comprising:
attaching the conjugate of claim 1 to a target cell;
subjecting said target cell attached to said conjugate to magnetic processing;
cleaving the peptide bond in said conjugate to separate said target cell from the magnetic particle in said conjugate; and
providing a substrate and allowing said target cell separated from said magnetic particle to attach to said substrate.

16. The method of claim 9, wherein said magnetic processing comprises magnetically sorting or separating cells.

17. A method of forming a conjugate for attachment to a cell, comprising:
linking a surface modifier to a magnetic particle with a linker to form the conjugate;
wherein said surface modifier is selected to have a specific affinity to said cell; and
wherein said linker is selected such that said linker comprises a protease recognition site and a peptide bond, and cleavage of said peptide bond is catalyzed by a specific protease that recognizes said protease recognition site.

18. The method of claim 17, wherein said protease is thrombin.

19. The method of claim 17, wherein said surface modifier comprises a glucosamine, glutamine, or galactose.

20. The method of claim 17, wherein said magnetic particle comprises a quantum dot or a nanoparticle.

21-25. (canceled)


SEQUENCE LISTING (7 sequences)

SEQ ID NO: 1 - protein, 6 aa, artificial
  synthetic
XXPXXX

SEQ ID NO: 2 - protein, 6 aa, artificial
  synthetic
LVPRGS

SEQ ID NO: 3 - protein, 6 aa, artificial
  synthetic
MYPRGN

SEQ ID NO: 4 - protein, 6 aa, artificial
  synthetic
IRPKLK

SEQ ID NO: 5 - protein, 10 aa, artificial
  synthetic
CLVPRGSGCG

SEQ ID NO: 6 - protein, 13 aa, artificial
  synthetic
LIVMSTASTAGHC

SEQ ID NO: 7 - protein, 7 aa, artificial
  synthetic
ENLYFQX